(19) United States
(12) Patent Application Publication
(10) Pub. No.: US 2025/0120847 A1
(43) Pub. Date: Apr. 17, 2025

(54) PORTABLE URINE COLLECTION SYSTEM AND RELATED METHODS

(71) Applicant: PUREWICK CORPORATION, Covington, GA (US)

(72) Inventors: Kuilin Lai, Watkinsville, GA (US); Puja Patel, Lawrenceville, GA (US); Sudhakar Jagannathan, Alpharetta, GA (US); Tacarra Gordon, Loganville, GA (US); Nicholas Jardine, Holly Springs, NC (US); Tamika Richardson, Schenectady, NY (US); Anthony Garvey, Listowel (IE); Padraig Fogarty, Ballina-Killaloe (IE); Meghan Caracciola, Washington, DC (US)

(21) Appl. No.: 18/681,987

(22) PCT Filed: Aug. 8, 2022
(86) PCT No.: PCT/US2022/039711

§ 371 Date: Feb. 7, 2024

Related U.S. Application Data
(60) Provisional application No. 63/230,894, filed on Aug. 9, 2021.

(51) Int. Cl. A61F 5/451; A61F 5/44

(52) U.S. Cl. CPC A61F 5/451; A61F 5/4408

(57) ABSTRACT

Examples relate to devices, systems, and methods for fluid collection. A fluid collection system may include a fluid collection device configured to collect fluid discharged from a user, a fluid collection container configured to receive the fluid from the fluid collection device, a pump in fluid communication with the fluid collection container configured to pull an at least partial vacuum on an interior region of the fluid collection container effective to draw fluid from the fluid collection device into the fluid collection container. The fluid collection container is either a reusable container or a disposable container. At least the pump and the disposable container are sized and dimensioned to be disposed within a portable carrying case.

### CROSS-REFERENCE TO RELATED APPLICATIONS

This application claims priority to U.S. Provisional Patent application No. 63/230,894 filed on Aug. 9, 2021, entitled PORTABLE URINE COLLECTION SYSTEM AND RELTED METHODS, the disclosure of which is incorporated by reference herein, in its entirety.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bedpans and urinary catheters have several problems associated therewith. For example, bedpans may be prone to discomfort, spills, and other hygiene issues. Urinary catheters be may be uncomfortable, painful, and may cause urinary tract infections. Conventional fluid collection devices also may be limited to use when a patient is confined to a bed in a supine position.

Thus, users and manufacturers of fluid collection devices continue to seek new and improved devices, systems, and methods to collect fluid.

### SUMMARY

Embodiments disclosed herein are related to fluid collection systems and methods of using fluid collection systems. In an embodiment, a fluid collection system may include a fluid collection device configured to collect fluid discharged from a user, a fluid collection container configured to receive the fluid from the fluid collection device, and a pump in fluid communication with the fluid collection container. The pump may be in fluid communication with the fluid collection container and configured to pull an at least partial vacuum on an interior region of the fluid collection container effective to draw fluid from a fluid collection device into the fluid collection container. The fluid collection container may be either a reusable container or a disposable container. In some embodiments, at least the pump and the disposable container are sized and dimensioned to be disposed within a portable carrying case.

In an embodiment, a portable fluid collection system may include a fluid collection device, a first tube in fluid communication with the fluid collection device, a fluid collection container including a rectangular shape, a pump in fluid communication with the fluid collection container, a power supply operatively coupled to the pump, and a drainage tube configured to couple to the fluid collection container. The fluid collection device may be configured to be positioned at least proximate to a urethra of a user. The pump may be configured to pull an at least partial vacuum on an interior region of the fluid collection container effective to draw fluid from the fluid collection device through the first tube into the fluid collection container.

In an embodiment, a method of assembly for a portable fluid collection system is disclosed. The method may include positioning a fluid collection device proximate to a urethra of a user. The method also may include fluidly coupling the fluid collection device to the fluid collection container with a first tube. The method may further include placing a pump in fluid communication with the fluid collection container and placing at least the pump, a battery, and the fluid collection container within a portable carrying case. The method may also include detachably coupling the portable carrying case to a wheelchair. The pump may be configured to pull an at least partial vacuum on the interior region of the fluid collection container effective to draw fluid from the fluid collection device through the first tube into the fluid collection container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a portable fluid collection system, according to an embodiment.

FIG. 2A is an isometric view of a fluid collection system, according to an embodiment.

FIG. 2B is an isometric view of a portable fluid collection system, according to an embodiment.

FIG. 3 is an isometric view of a fluid collection container, according to an embodiment.

FIG. 4A is a rear view of a portable carrying case for a fluid collection system, according to an embodiment.

FIG. 4B is a cross sectional view of a portable fluid collection system and carrying case, according to an embodiment.

FIG. 5A is a rear isometric view of a fluid collection system including an alternator coupled to a wheelchair, according to an embodiment.

FIG. 5B is a front isometric view of a fluid collection system including a pump coupled to a wheelchair, according to an embodiment.

FIG. 5C is a schematic view of a pump, according to an embodiment.

FIG. 6 is a schematic view of a filter system for a fluid collection system, according to an embodiment.

FIG. 7 is a flow diagram of a method for assembling a portable fluid collection system, according to an embodiment.

### DETAILED DESCRIPTION

Embodiments disclosed herein are related to fluid collection systems and related methods. Many users of fluid collection devices are over 65 years old with limited mobility, often relying on wheelchairs as a primary mode of transportation. Many users also spend a significant amount of their day in a seated or supine position. Users and caregivers, then, are benefited from a fluid collection system that may be both discrete and mobile, allowing users to use the fluid collection system to collect fluid both at home and on the go.

In many embodiments described herein, a fluid collection system may be configured to be capable for extended use at home or may be relatively compact and configured to be portable. Embodiments of the fluid collection systems described herein are both able to include large capacity of urine collection for at-home (e.g., overnight) use and mobile and discreet, allowing a user to participate in social activities without alerting others to the incontinence of the users. For example, the fluid collection systems may include a portable carrying case configured to detachably couple to a wheelchair and hold at least a pump of a fluid collection system therein to obscure the pump from view and/or dampen noise outside the fluid collection system. The fluid collected in the fluid collection systems described herein also may be stored in a fluid collection container that is obscured by the portable carrying case from view and/or obscures the fluid held in the fluid collection container. Portable can be defined as able to be easily carried or moved, especially because being of a lighter and smaller version than fluid collection systems that include large capacity of urine collection. In other words, the portable carrying case can be carryable by a human, such as a caretaker or a patient.

In some embodiments, the fluid collection system also includes a fluid collection device configured to be positioned at least proximate to a urethra of a user and a first tube in fluid communication with the fluid collection device where the pump may be configured to pull an at least partial vacuum on an interior region of the fluid collection container effective to draw fluid from the fluid collection device through the first tube into the fluid collection container.

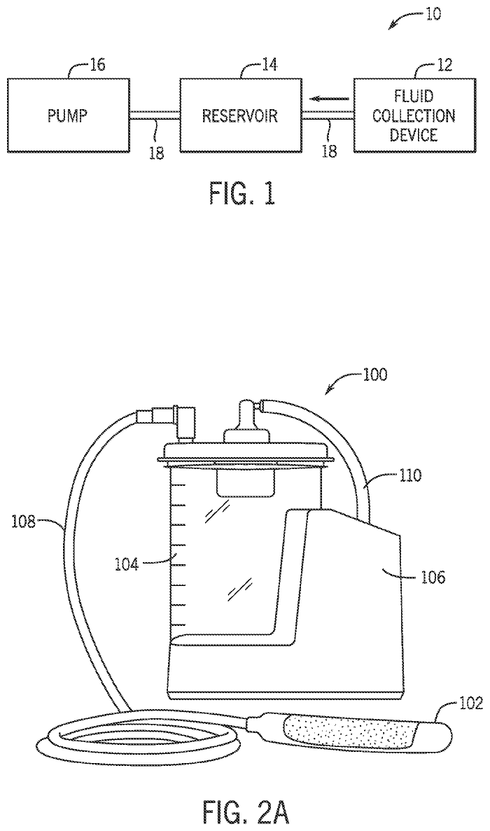

FIG. 1 is a block diagram of a fluid collection system 10, according to an embodiment. The fluid collection system 10 may be included in embodiments of fluid collection systems described herein. The system 10 includes a fluid (e.g., urine) collection device 12 (e.g., any of the fluid collection assemblies disclosed herein), a fluid collection container 14 (or reservoir), and a pump 16 (or portable vacuum device). The fluid collection device 10, the fluid collection container 14, and the pump 16 may be fluidly coupled to each other via one or more tubes 18. For example, fluid collection device 10 may be operably coupled to one or more of the fluid collection container 14 or the pump 16 via the tube 18. In some embodiments, the pump 16 may be coupled directly to the fluid collection container 14. Fluid (e.g., urine or other bodily fluids) collected in the fluid collection device 10 may be removed from the fluid collection device 10 via the tube 18 coupled to the fluid collection device 12. Suction force may be introduced into the chamber of the fluid collection device 12 via the inlet of the tube 18 responsive to suction (e.g., vacuum) force applied at the outlet of the tube 18.

The suction force may be applied to the outlet of the tube 18 by the pump 16 either directly or indirectly. The suction force may be applied indirectly via the fluid collection container 14. For example, the outlet of the tube 18 may be disposed within or fluidly coupled to an interior region of the fluid collection container 14 and an additional tube 18 may extend from the fluid collection container 14 to the pump 16. Accordingly, the pump 16 may apply suction to the fluid collection device 12 via the fluid collection container 14. The suction force may be applied directly via the pump 16. For example, the outlet of the tube 18 may be disposed within the pump 16. An additional tube 18 may extend from the pump 16 to a point outside of the fluid collection device 12, such as to the fluid collection container 14. In such examples, the pump 16 may be disposed between the fluid collection device 12 and the fluid collection container 14.

The fluid collection container 14 may be sized and shaped to retain a fluid therein. The fluid collection container 14 may include a bag (e.g., drainage bag), a bottle or cup (e.g., collection jar), or any other enclosed container for storing bodily fluid(s) such as fluid. In some examples, the tube 18 may extend from the fluid collection device 12 and attach to the fluid collection container 14 at a first point therein. An additional tube 18 may attach to the fluid collection container 14 at a second point thereon and may extend and attach to the pump 16. Accordingly, a vacuum (e.g., suction) may be drawn through fluid collection device 12 via the fluid collection container 14. Fluid, such as urine, may be drained from the fluid collection device 12 using the pump 16.

The pump 16 or vacuum source may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), an alternator, one or more batteries, or even manual power (e.g., a hand operated vacuum pump). The vacuum sources disclosed herein may include one or more of a switch, a button, a plug, a remote, or any other device suitable to activate the pump 16.

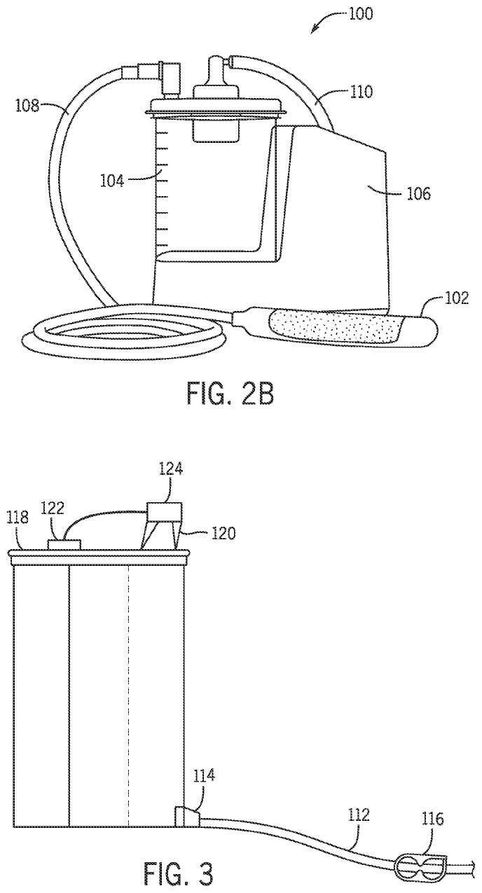

Many embodiments of fluid collection systems described herein are configured to be worn by a user, positioned on a surface such as a table, and/or securable or mountable to a wheelchair. FIGS. 2A-2B is an isometric view of a fluid collection system 100, according to some embodiments. The fluid collection system 100 may include a fluid collection device 102 configured to receive fluid (e.g. urine) from a user. The fluid collection assembly 102 may be removably attached to a fluid collection container 104 of the fluid collection system 100. The fluid collection container 104 may be integrated into a housing 106 that may also include a pump therein. The fluid collection system may further include a collection tube 108 and a suction tube 110. The collection tube 108 may be disposed between the fluid collection device 102 and the fluid collection container 104. The collection tube 108 may fluidly connect the internal volume of the fluid collection container 104 with the fluid collection assembly 102. The suction tube 110 may couple the pump suction and/or discharge to the fluid collection container 104. The pump disposed within the housing 106 may be in fluid communication with the internal volume of the fluid collection container 104 via the suction tube 110. The housing 106 may also be configured to store or house internal electrical and mechanical components. For example, the housing may include a pump (not shown in FIG. 2A) configured to modulate pressure within the container 104.

The fluid collection device 102 may be positioned at least proximate to a urethral opening. The fluid collection device shown in FIGS. 2A-2B is an example of a female fluid collection assemblies configured to collect fluid(s) from females (e.g., collect urine from a female urethra). Further examples of female fluid collection assemblies are disclosed in U.S. Pat. No. 10,390,989 issued on Aug. 27, 2019, the disclosure of which is incorporated herein, in its entirety, by this reference. However, the fluid collection assemblies, systems, and methods disclosed herein may include male fluid collection assemblies and/or devices shaped, sized, and otherwise configured to collect fluid(s) from males (e.g., collect urine from a male urethra). Examples of male fluid collection assemblies are disclosed in U.S. Provisional Patent Applications No. 63/067,542 filed on Aug. 19, 2020 and U.S. patent application Ser. No. 16/433,773 filed on Jun. 6, 2019, the disclosure of which are incorporated herein, in its entirety, by this reference.

In some embodiments, the collection tube 108, the suction tube 110 and/or other tubing of the fluid collection system 100 may include a flexible material such as materials tubing (e.g., medical tubing). Such material tubing may include a thermoplastic elastomer, polyvinyl chloride, ethylene vinyl acetate, polytetrafluoroethylene, flexible metal, ceramic and composite material tubing etc. The collection tube 108 may include silicon or latex. In some embodiments, the collection tube 108 may be constructed of any suitable material to be impermeable to fluids such that fluids may be drawn from the fluid collection assembly 102 and into the collection tube 108. In some embodiments, the collection tube 108 may include one or more portions that are resilient, such as by having one or more of a diameter or wall thickness that allows the collection tube 108 to be flexible.

The fluid collection container 104 may be integrated into the housing 106. In some embodiments, the container 104 may include at least an inlet coupled to the collection tube 108, an outlet coupled to a drain tube and a vacuum port coupled to the pump. In some embodiments, the container 104 may be opaque or clear according to different embodiments and may include a generally rectangular front or rear profile as shown in FIGS. 2A-2B. As shown in FIG. 2A, in some embodiments, the fluid collection container 104 may include a large capacity from about 1000 mL to about 2000 mL. The fluid collection container 104 may include the large capacity for home use, where the fluid collection system 100 may be used for a long duration, without needing to refill and/or exchange the fluid collection container 104. Uses for the large capacity fluid collection container 104 may include travelling long distances, overnight use, and times when a caregiver may not be able to attend the user and/or empty/replace the fluid collection container 104.

As shown in FIG. 2B, in some embodiments, the fluid collection container 104 may include a relatively smaller capacity from about 100 mL to about 500 mL. Uses for the smaller capacity fluid collection container 104 may include portability, discretion, less weight and more convenient size of the fluid collection system 100, and improved independence for the user. The fluid collection container 104 may be rectangular shaped. The rectangular shape may improve the portability of the fluid collection system 100 by making the system easier to put into a portable carrying case (e.g. a backpack). In some embodiments, the fluid collection container 104 may be reusable. The user and/or caretaker may empty, clean, and replace the fluid collection container 104. In other embodiments, the fluid collection container 104 may be disposable. The fluid collection container 104 may be thrown away and a replacement container installed.

FIG. 3 illustrates an embodiment of fluid collection container 104. The fluid collection container 104 may include a durable plastic canister and may include a capacity from about 1000 mL to about 2000 mL or from about 100 mL to about 500 mL. The fluid collection container 104 may include a rectangular shape to allow the container 104 to securely and discretely fit within a portable carrying case. The fluid collection container 104 may be reusable and dishwasher safe, and may include a generally rigid material such as polycarbonate, plastic, rubber, metal, glass, combinations thereof, or any other suitable materials. In other embodiments, the fluid collection container 104 may be disposable and biodegradable.

In some embodiments, the fluid collection container 104 may include a drain tube 112 to remove fluid from the fluid collection container 104. Fluid collected in the fluid collection container 104 may be emptied through the drain tube 112. The drain tube 112 may be coupled to a drain port 114. In some embodiments, the drain port 114 may be located in a bottom corner of the fluid collection container 104. In some embodiments, the drain port 114 may automatically open when the drain tube 112 couples to the drain port 114. The drain port 114 may include a valve that opens when the drain tube 112 is coupled to the drain port 114. For example, the drain port 114 may include a split septum device that opens when the drain tube 112 is inserted. In other embodiments, the drain port 114 may be a one-way valve that is activated open when the drain tube 112 is connected.

In some embodiments, the drain tube 112 may include a valve 116 that may be opened to empty the fluid collection container 104 directly or a pump may be coupled to the fluid collection container 104 and configured to apply a pressure to the fluid for more rapid disposal. The valve 116 may include a clamp, a ball valve, a butterfly valve, or any suitable valve.

In some embodiments, the fluid collection container 104 includes a lid 118 and a drain spout 120 and vent spout 122 coupled to the lid 118. In some embodiments, the lid 118 further includes connectors (not shown) for the collection tube 108 and the suction tube 110. The drain spout 120 may include a cap 124 that covers the drain spout 120 when not in use. The cap 124 may be connected to a cover (not shown) for the vent spout 122 and configured such that the cap 124 is removed and the vent spout 122 is open when the fluid is dispensed, to aid in proper air flow as the fluid is removed.

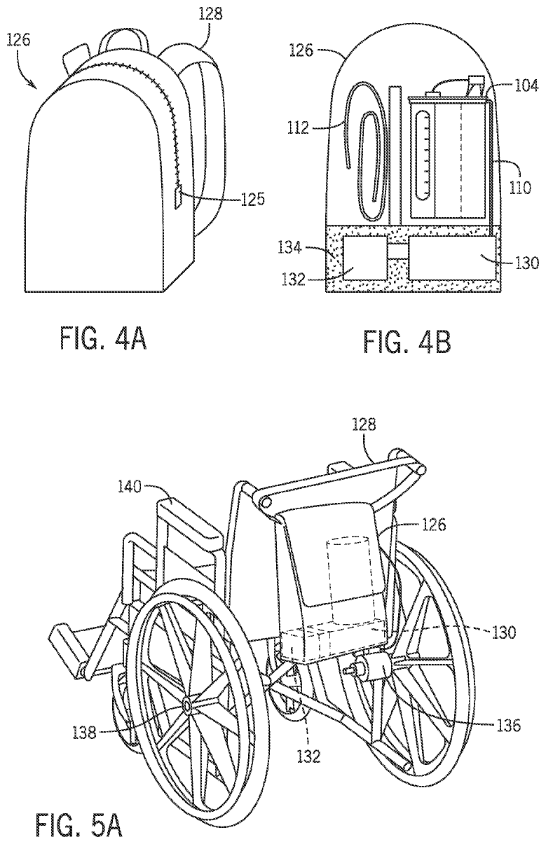

Referring now to FIGS. 4A-4B, the fluid collection system 100 may include a portable carrying case 126 that may be worn by a user and/or a caregiver. In some embodiments, the portable carrying case 126 may include one or more straps 128 to couple the portable carrying case 126 to at least one of the user, one or more wheelchair handles, a bed, or a table. In some embodiments, an outer surface of the portable carrying case 126 may include Velcro or other fasteners configured to couple directly to a wheelchair. The portable carrying case 126 may be a generally rectangular shape with a flat bottom for stability and for being able to fit underneath a wheelchair and/or coupled to a back of the wheelchair. The one or more straps 128 may be configured to be worn on a shoulder or to detachably secure, mount, or hang from a stationary or mobile structure (e.g., a wheelchair) and support the fluid collection system 100. In some embodiments, the portable carrying case 126 may include at least one of a backpack or shoulder bag. In some embodiments, the portable carrying case 126 may include a durable outer cover and/or shell.

In some embodiments, the portable carrying case 126 may include a fastening seam 125, such as a hook and loop, snap, or zipper seam. In some embodiments, the portable carrying case 126 may include more than one compartments, each compartment being able to be accessed by a fastening seam. Thus, the portable carrying case 126 may be closeable for discretion of the fluid collection system 100 and may be used to hold other items securely. For example, when the drain tube 112 is not being used, the drain tube 112 may be stored within the portable carrying case 126. The portable carrying case 126 may be sized and dimensioned to hold at least the fluid collection container 104, a pump 130, and a battery 132 (shown in FIG. 4B) therein.

The pump 130 may be in fluid communication with the interior region of the fluid collection container 104 and may be configured to pull at least a partial vacuum on the interior region of the fluid collection container 104 effective to draw the fluid from the fluid collection device 102 and into the fluid collection container 104. In some embodiments, the pump 130 may be coupled directly to the fluid collection container 104, or the suction tube 110 may fluidly couple the pump 130 with the interior region of the fluid collection container 104.

The pump 130 may include one or more of a manual vacuum pump, and electric vacuum pump, a diaphragm pump, a centrifugal pump, a displacement pump, a magnetically driven pump, a peristaltic pump, or any pump configured to produce a vacuum. For example, the pump may include an air media diaphragm pump having a minimum pumping speed of 25 ml/second. In some embodiments, the pump 130 includes a variable speed pump and/or a continuous pump. For example, the pump 130 may include a variable speed pump. The pump 130 may provide a vacuum or suction to remove fluid from the fluid collection device 102. In some examples, the pump 130 may be powered by one or more batteries 132 operatively coupled to the pump. In some embodiments, the battery 132 may include a lithium ion battery. In some embodiments, the battery 132 may be alkaline or rechargeable. In some examples, the pump 130 and/or the battery 132 may be sized and shaped to fit within the portable carrying case 126. For example, the pump 130 may include one or more miniaturized pumps or one or more micro pumps. The pump 130 may include one or more of a switch, a button, a plug, a remote, or any other device suitable to activate the pump 130.

As shown in FIG. 4B, the portable carrying case 126 may include a sound dampening material 134 where the pump 130 and/or the battery 132 may be located. In some embodiments, the sound dampening material 134 may be configured to dampen the operational sound of the pump 130 and secure the pump 130 and/or the battery 132, and/or other components within the portable carrying case 126. The sound dampening material 134 may include sound-deadening foam. Sound-deadening foam may include egg crate style foam constructed of open cell melamine, polyurethane, or other suitable foam material. The sound dampening material 134 may include a composite of synthetic or natural materials. In addition to abating sound, the sound dampening material 134 may minimize operational vibrations of the fluid collection system 100.

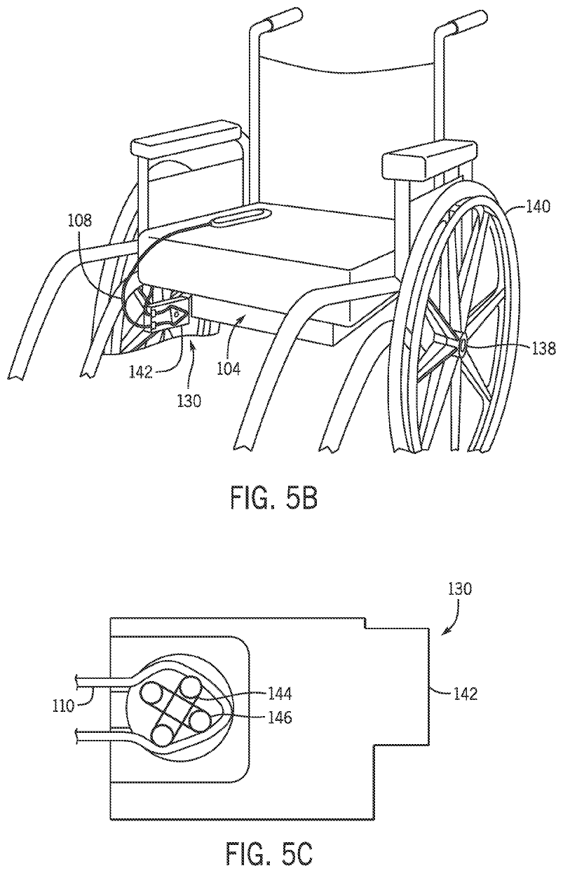

Referring now to FIG. 5A, in some embodiments, the fluid collection system 100 may be powered by the battery 132 and may also include an additional power supply. The additional power supply may be used when the battery 132 is at a low power status or charging. In some embodiments, the additional power supply may include an outlet on an electric powered wheelchair. Thus, the fluid collection system 100 may couple to a receptacle on the electric powered wheelchair. In some embodiments, the fluid collection system 100 may include any suitable connector to electrically couple the fluid collection system 100 to the additional power supply (e.g., USB port, Neutrik connector, XLR connector, A/C D/C converter, 3-pin charge socket, etc.).

In some embodiments, the fluid collection system 100 may include an alternator 136 to charge the battery 132 or power an electric motor for the pump 130. In some embodiments, the alternator 136 may be coupled to an axle 138 of the wheelchair 140. When the wheelchair is moved, the alternator 136 may be coupled to the wheel to generate a current that may charge the battery 132 and/or power the pump 130. The alternator 136 may extend the life of the battery 132 in operation by powering the components of the fluid collection system 100 (e.g. the pump 130) directly when the wheelchair 140 is moving. In some embodiments, the alternator 136 may charge the battery 132 and operate the pump 130 or other component of the fluid collection system 100 simultaneously.

Referring now to FIGS. 5B-5C, in some embodiments, the pump 130 may be powered directly by the motion of the wheelchair 140. In some embodiments, the pump 130 may include a peristaltic pump 142. The peristaltic pump 142 may include a roller portion 144 configured to compress a tube (e.g., collection tube 108 or suction tube 110) to draw fluid from the fluid collection device 102 to the fluid collection container 104. In FIG. 5B, the fluid collection container 104 is shown underneath a seat of the wheelchair 140. The roller portion 144 of the peristaltic pump 142 may be coupled directly to the axle 138 of the wheelchair 140. For example, when the wheelchair 140 moves forward the rotating axle 138 of the wheelchair 140 rotates the roller portion 144 of the peristaltic pump 142. In some embodiments, the roller portion 144 may be directly coupled to the axle 138. In other embodiments, the roller portion 144 may be coupled to a gear system (not shown) that establishes a gear ratio between the axle 138 and the roller portion 144 of the peristaltic pump 142. In some embodiments, the roller portion 144 may operate on a ratchet gear, such that when the wheelchair wheels, roll backwards, the roller portion 144 does not rotate in a reverse direction in order to prevent urine from flowing back to the fluid collection device 102.

The peristaltic pump 142 may include a second roller portion 146, in some embodiments. The roller portion 144 discussed above may be mechanically coupled to the axle 138 and the second roller portion 146 may be electrically coupled to the battery 132 or an alternate power supply discussed above. Thus, in some embodiments, the roller portion 144 of the pump 142 may be configured to receive energy from the mechanical motion of the wheel chair 140 to pump fluid through the peristaltic pump 142 and the second roller portion 146 may be configured to receive electrical energy from the battery 132 or alternate electrical power supply to pump fluid. In some embodiments, the roller portions 144 and 146 are configured to operate independently and/or synchronously. For example, when the wheelchair 140 is moving forward, the pump 130 may operate solely with the roller portion 144 to transfer fluid. When the wheelchair 140 is stopped or moving backwards, the pump 130 may operate only the second roller portion 146, and be powered by the battery 132, alternator 136, or other power supply.

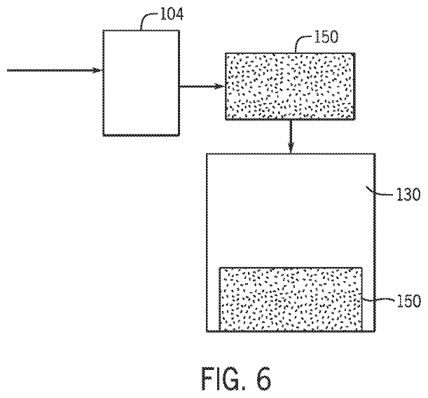

Referring now to FIG. 6, in some embodiments, the fluid collection system 100 may include one or more odor filters 150 configured to at least partially filter or neutralize an odor of the fluid. The filter 150 may be configured to neutralize odor of the air being pulled from the interior region of the fluid collection container 104 by the pump 130. In some embodiments, the filter 150 may be positioned between at least a portion of the collection tube 108 and a portion of the interior region of the fluid collection container 104 such that air being pulled from the interior region of the fluid collection container 104 may be filtered before or as the air enters the suction tube 110. In some embodiments, the filter 150 may be positioned on an exhaust vent on the pump 130. In some embodiments, a filter 150 may be positioned both at the exhaust vent on the pump 130 and before air enters the suction tube 110. The filter 150 may include an odor-absorbing filter and/or a hydrophobic filter configured to prevent or minimize fluid from the fluid collection container 104 being pulled into the pump 130.

In some embodiments, the filter 150 may include an activated carbon filter. In other embodiments, the filter 150 may include a gas filter. In some embodiments, the one or more odor filter 150 may be included on or within at least one of the fluid collection device 102, the portable carrying case 126, or the fluid collection container 104.

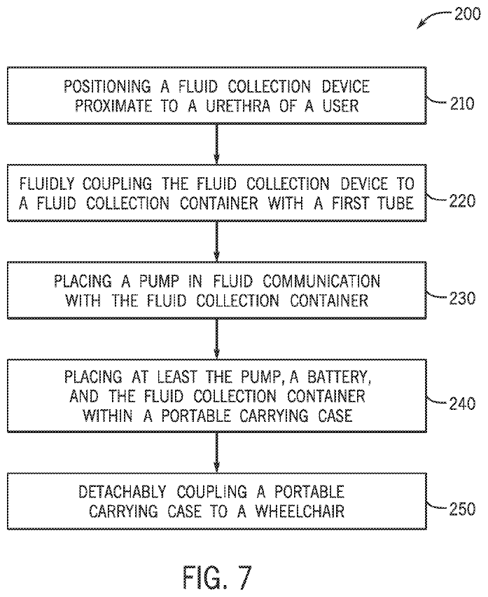

FIG. 7 is a flow diagram of a method 200 for assembling a portable fluid collection system, according to an embodiment. The method 200 includes an act 210 of positioning a fluid collection device proximate to a urethra of a user and an act 220 of fluidly coupling the fluid collection device to a fluid collection container with a first tube. The method 200 also includes an act 230 of placing a pump in fluid communication with the fluid collection container. The pump may be configured to pull a vacuum on the interior region of the fluid collection container effective to draw fluid from the fluid collection device through the first tube into the fluid collection container. The method 200 may also include an act 240 of placing at least the pump, a battery, and the fluid collection container within a portable carrying case and an act 250 of detachably coupling a portable carrying case to a wheelchair.

The method 200 may include assembling any of the fluid collection system embodiments described herein. For example, the act 250 of detachably coupling a portable carrying case to a wheelchair may include coupling one or more straps of the portable carrying case to one or more handles of the wheelchair. The fluid collection container may include either a reusable container or a disposable container. In some embodiments, the method of assembly the portable fluid collection system includes cleaning and replacing the container or disposing of the container and fluidly coupling a new fluid collection container to the fluid collection device. In some embodiments, the method of assembly the portable fluid collection system may include energizing the pump with an alternated operatively coupled to an axle of the wheelchair.

The acts of the method of collecting fluids from a user described above are for illustrative purposes. For example, the acts of the method of collecting fluids from a user can be performed in different orders, split into multiple acts, modified, supplemented, or combined. In an embodiment, one or more of the acts of the method of collecting fluids from a user can be omitted from the method. Any of the acts of the method of collecting fluids from a user can include using any of the portable fluid collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A fluid collection system, comprising:
a fluid collection device configured to collect fluid discharged from a user;
a fluid collection container configured to receive the fluid from the fluid collection device;
a pump in fluid communication with the fluid collection container configured to pull an at least partial vacuum on an interior region of the fluid collection container effective to draw fluid from a fluid collection device into the fluid collection container; and
a drainage tube configured to be coupled to the fluid collection container, wherein the pump de-energizes when the drainage tube is coupled to the fluid collection container;
wherein the fluid collection container is either a reusable container or a disposable container; and
wherein at least the pump and the disposable container are sized and dimensioned to be disposed within a portable carrying case.

2. The fluid collection system of claim 1, wherein the portable carrying case includes a sound dampening material, wherein at least the pump is disposed within the sound dampening material.

3. The fluid collection system of claim 2, wherein the sound dampening material includes a foam material configured to dampen operational sound of at least the pump.

4. The fluid collection system of claim 1, wherein the reusable container includes a capacity from about 1000 mL to about 2000 mL.

5. The fluid collection system of claim 1, wherein the disposable container includes a capacity from about 100 mL to about 500 mL.

6. The fluid collection system of claim 1, wherein the reusable container and the disposable container are rectangular.

7. The fluid collection system of claim 1, wherein the pump includes a peristaltic pump.

8. The fluid collection system of claim 7, wherein the peristaltic pump includes a roller portion configured to compress a tube, wherein the roller portion is coupled to a wheel axle of a wheelchair.

9. The fluid collection system of claim 7, wherein the peristaltic pump includes a first roller portion operatively coupled to a wheel axle of a wheelchair and a second roller portion operatively coupled to an electric motor, wherein the first roller portion and the second roller portion are configured to rotate independently.

10. The fluid collection system of claim 1, further comprising an odor filter including at least one of an activated carbon filter or a gas filter.

11. A portable fluid collection system, comprising:
a fluid collection device configured to be positioned at least proximate to a urethra of a user;
a first tube in fluid communication with the fluid collection device;
a fluid collection container including a rectangular shape;
a pump in fluid communication with the fluid collection container and configured to pull an at least partial vacuum on an interior region of the fluid collection container effective to draw fluid from the fluid collection device through the first tube into the fluid collection container;
a power supply operatively coupled to the pump; and
a drainage tube configured to couple to the fluid collection container, wherein the pump de-energizes when the drainage tube is coupled to the fluid collection container.

12. The portable fluid collection system of claim 10, wherein the fluid collection system is sized and dimensioned to be disposed within a portable carrying case.

13. The portable fluid collection system of claim 11, wherein the drainage tube includes a valve that opens to drain the fluid collection container.

14. (canceled)

15. The portable fluid collection system of claim 11, further comprising a lid for the fluid collection container, the lid including a dispensing spout and a vent.

16. The portable fluid collection system of claim 11, wherein the fluid collection system includes one or more odor filters configured to at least partially filter or neutralize an odor of the fluid.

17. The portable fluid collection system of claim 11, wherein the portable fluid collection system further includes an alternator coupled to a wheelchair wheel configured to charge the battery when the wheelchair wheel rotates.

18. The portable fluid collection system of claim 12, wherein the portable carrying case includes one or more compartments sized and dimensioned to hold at least the drainage tube when the drainage tube is not coupled to the fluid collection container.

19. A method of assembling a portable fluid collection system, the method comprising:
positioning a fluid collection device at least proximate to a urethra of a user;
fluidly coupling the fluid collection device to a fluid collection container with a first tube;
placing a pump in fluid communication with the fluid collection container configured to pull an at least partial vacuum on the interior region of the fluid collection container effective to draw fluid from the fluid collection device through the first tube into the fluid collection container;
placing at least the pump, a battery, and the fluid collection container within a portable carrying case; and
detachably coupling the portable carrying case to a wheelchair and energizing the pump with an alternator operatively coupled to an axle of the wheelchair.

20. The method of claim 19, wherein the fluid collection container includes either a reusable container or a disposable container.

21. (canceled)
